(19) United States
(12) Patent Application Publication
(10) Pub. No.: US 2024/0016246 A1
(43) Pub. Date: Jan. 18, 2024

(54) NEAR FIELD SAFE ANTIMICROBIAL WAVELENGTH DISINFECTION VIA OPTICAL FIBERS, LIGHT GUIDES, OR LIGHT PIPES FOR PERCUTANEOUS OR INDWELLING AMBULATORY DISINFECTION OF CATHETERS, VASCULAR ACCESS LINES, DRAINS, ENDO-TRACHEAL TUBES AND SIMILAR MEDICAL DEVICES

(71) Applicants: Sharon A. Keene, Tucson, AZ (US); Erik Shahoian, Sonoma, CA (US)

(72) Inventors: Sharon A. Keene, Tucson, AZ (US); Erik Shahoian, Sonoma, CA (US)

(21) Appl. No.: 18/224,511

(22) Filed: Jul. 20, 2023

Related U.S. Application Data
(63) Continuation-in-part of application No. PCT/US23/25626, filed on Jun. 16, 2023.
Application No. PCT/US23/25626 is a continuation-in-part of application No. 17/952,139, filed on Sep. 23, 2022, now abandoned.
Application No. 17/952,139 is a continuation-in-part of application No. 17/874,178, filed on Jul. 26, 2022, now abandoned.
Application No. 17/874,178 is a continuation-in-part of application No. 17/843,914, filed on Jun. 17, 2022.
Application No. 17/843,914 is a continuation-in-part of application No. 17/503,151, filed on Oct. 15, 2021, now Pat. No. 11,425,945.
Application No. 17/503,151 is a continuation-in-part of application No. 17/503,153, filed on Oct. 15, 2021, now Pat. No. 11,412,792.
(60) Provisional application No. 62/994,523, filed on Mar. 25, 2020.
(60) Provisional application No. 63/143,677, filed on Jan. 29, 2021.

(51) Int. Cl. A41D 13/11; A61L 2/10

(52) U.S. Cl. CPC A41D 13/1192; A41D 13/1107; A61L 2/10; A61L 2202/20; A61L 2202/11

(57) ABSTRACT

Far UV-C wavelengths provide safe and effective antimicrobial disinfectants without genotoxic or ocular toxicities associated with broadband UV-C disinfection. Antimicrobial wavelengths must be applied judiciously to avoid dysbiosis of human and environmental microbiomes. Disclosed is the application of far UV-C or other safe antimicrobial wavelengths via optical fiber delivery to disinfect percutaneous or indwelling medical devices such as various medical grade catheters (urinary, IV), tubes or drains which are a primary source of nosocomial infection. A central photon source is configured to deliver disinfecting antimicrobial wavelengths into one or more optical fibers, light pipes or light guides embedded or molded into such devices to distribute disinfecting wavelengths near field to specific animate and inanimate targets for optimal efficacy with limited, radiant dosing minimizing antimicrobial exposure of non-targeted surfaces to reduce or eliminate infectious pathogens, reduce nosocomial infection while minimizing impact on human and environmental microbiomes.

### CROSS REFERENCE TO RELATED APPLICATIONS

This application is a continuation-in-part of PCT application PCT/US2023/25626 filed Jun. 16, 2023, which in turn claims benefit to U.S. patent application Ser. No. 17/952,139, filed Sep. 23, 2022, which application in turn is a continuation-in-part of U.S. patent application Ser. No. 17/874,178, filed Jul. 26, 2022, which application in turn is a continuation-in-part of U.S. patent application Ser. No. 17/843,914, filed Jun. 17, 2022, which application in turn is a continuation-in-part of divisional application of U.S. patent application Ser. No. 17/503,151, now U.S. Pat. No. 11,425,945, granted Aug. 30, 2022, and Ser. No. 17/503,153, now U.S. Pat. No. 11,412,792, granted Aug. 16, 2022, both filed Oct. 15, 2021, which in turn, claim priority to U.S. patent application Ser. No. 17/169,253, filed Feb. 5, 2021, now U.S. Pat. No. 11,266,189, granted Mar. 8, 2022, which application in turn claims priority to U.S. Provisional Patent Application Ser. No. 62/994,523, filed Mar. 25, 2020 and U.S. Provisional Patent Application Ser. No. 63/143,677, filed Jan. 29, 2021, the contents of which are incorporated herein by reference.

### BACKGROUND AND SUMMARY

The present disclosure relates generally to near field application of safe antimicrobial wavelengths for disinfection of percutaneous or indwelling medical devices while they are in use to curb nosocomial infections. The disclosure has particular utility with respect to either immediate or long term maintenance of disinfection or sterilization of percutaneous or indwelling medical devices and adjacent potentially contaminated tissues, such devices include catheters, drains, vascular access devices, trocars and endotracheal or tracheostomy tubes, and will be described in connection with the use of near field application of safe antimicrobial wavelengths such as far UVC (200-230 nm) to maintain disinfection of such medical devices while simultaneously disinfecting or sterilizing surrounding animate and inanimate areas that serve as a source of contamination or infection.

Nosocomial (hospital-acquired) infections are a leading cause of extended hospital stays, increased hospitalization costs, and unnecessary morbidity and mortality in the United States. In addition to pandemic pathogens, such as COVID, a primary source of nosocomial infection involves ambient bacteria, fungi, virus and other pathogens which invade areas of a patient's body via medical devices such as percutaneous or indwelling catheters accessing various body cavities or spaces, vascular access lines, endotracheal or tracheostomy tubes, trocars and surgical drains to cause infection. However, in the latter case, drains may be placed for drainage of suspected contaminated fluids, in these cases antimicrobial light can be used to disinfect already contaminated tissues as well as the drains being used to reduce the pathogen burden.

In the aforesaid parent applications, the novel near field application of far UV-C wavelengths (200-230 nm) or other safe, visible antimicrobial wavelengths is described to simultaneously disinfect face masks/face shields and persons wearing them, as well as to simultaneously disinfect other personal protective equipment and various appliances and inanimate or animate fomite surfaces while such equipment or appliances are in use (ambulatory). For many decades broadband UV light and in particular broadband UVC wavelengths (100-280 nm) have been used as part of an effective antimicrobial disinfection strategy in hospitals and clinics, both for air and surface disinfection. Such disinfection has been established as superior to many topical disinfectants. However, broadband UVC light has limitations for use in the presence of people or animals because longer wavelengths within this group, the most popular being 254 nm, are genotoxic and damaging to a patient's skin and eyes and therefore pose health risks to humans or animals with direct exposure. In contrast, a narrow band of UVC light termed far UV-C light (200-230 nm) maintains the antimicrobial properties of broadband UVC, but exhibits the distinct advantage of being safe for exposure in humans or animals because its wavelengths are too short to be absorbed by the epidermis or even the tears of the eye, making it safe for exposure of skin and eyes of patients and healthcare workers that care for them. The near field application of such disinfecting wavelengths is maximized in this invention based on the property of light known as the inverse square law where antimicrobial radiance is stronger and more effective when applied in a near field distance (0-100 cm) from the photon source to a target which is advantageously achieved with devices described in this invention. Far UV-C light as well as certain other antimicrobial wavelengths of visible light can be delivered into small devices and tiny spaces such as IV catheters via optical fibers, or light pipes, or light guides to advantageously disinfect percutaneous areas and indwelling medical devices such as catheters, vascular access lines, endotracheal or tracheostomy tubes, surgical drains and trocars and surrounding skin or adjacent potentially contaminated tissues or to decontaminate them after an inadvertent contaminated manipulation of such a device, safely without damaging the skin or other tissues. The ability to deliver antimicrobial wavelengths in a near field location, which in this particular application is preferentially <10 cm and even more preferentially <1 cm, is advantageous for efficacy, as well as to limit the dose of light (power density (mW/cm2)×time×frequency of application) needed to deactivate a target pathogen and maintain disinfection at the intended target site. Reducing the need for greater time or frequency of antimicrobial wavelengths is advantageous for sparing the antimicrobial impact on non-targeted human or environmental surfaces in order to preserve critically important and healthy human or environmental microbiomes. Microbiomes refers to the plethora of useful bacteria, virus, and fungi or similar microbiota and their genomes that maintain health and wellness and also mediate various antibiotic resistance mechanisms.

As described in my parent applications nosocomial infections especially caused by pandemic respiratory pathogens such as SARS CoV 2 or COVID can significantly be relieved with effective control of infectious respiratory aerosols using near field antimicrobial light disinfection of a face mask/face shield designed to deactivate such pathogens on or in a mask or face shield as well as on a patients own skin additionally such safe antimicrobial wavelengths such as far UV-C (200-230 nm) are effective for simultaneous disinfection of frequently contaminated animate and inanimate fomite surfaces in the healthcare setting using near field application (within 100 cm, preferentially within 10 cm, and more preferentially <1 cm) in an ambulatory setting, simultaneously deactivating both animate and inanimate sources of contamination while surfaces are being touched or used, to deactivate pathogens before they cause infection. However, in addition to pandemic respiratory pathogens in the healthcare setting, other nosocomial infections are largely driven by bacterial, fungi, or viral pathogen contamination of percutaneous surfaces and subsequently indwelling medical devices, worn or used in the care of patients such apparatus provides surfaces and interfaces identified as primary infectious sources where such pathogens ascend from adjacent contaminated patient surfaces along the apparatus or device wall, or via inadvertent contamination from manipulation by a health care worker where such pathogens may invade the body in areas that should remain sterile or where presence of the particular pathogen disrupts the local microbiome and incites infection. Among such devices are urinary, spinal, or peritoneal catheters or tubes, indwelling vascular access lines such as intravenous or arterial lines, dialysis catheters (vascular or peritoneal), endotracheal or tracheostomy or chest tubes, feeding tubes, trocars and surgical drains into body cavities or subcutaneous spaces each of which represent access points for pathogens to enter a patient's body or have been placed in a potentially already contaminated cavity and where pathogen entry or multiplication of non-resident organisms can create infection. Reducing nosocomial infections with applicant's invention can substantially reduce patient's morbidity and mortality while saving billions of dollars in health care costs to hospitals and healthcare systems.

Studies have documented a common point for infectious access to the body is the interface between the percutaneous entry for indwelling medical devices such as catheters, access lines, drains and trocars and a patient's skin or other adjacent animate surfaces that may harbor bacterial contaminants or other pathogens (e.g. the urethral orifice for urinary catheters), whereby the catheters, access lines, drainage or feeding tubes and trocars act as access points for pathogens to migrate and invade a sterile physiologic system such as the urinary bladder or blood-stream or intestinal tract or tissues traversed by a surgical drain; or in the case of an endotracheal or tracheostomy tube, allows transfer of a pathogen that is not a normal inhabitant of the pulmonary microbiome. For example, ascending infection into the urinary bladder from the outer wall of the urinary catheter as it enters the urethra reportedly is the number one source for nosocomial urinary tract infection. Similarly, the percutaneous skin entrance where a catheter traverses the skin and enters a blood vessel for vascular access lines, is a location where natural microbial inhabitants or pathogenic contaminants can migrate along the exposed wall of the vascular catheter into the body which can result in serious systemic infection. Similar pathogen migration also can occur along exposed surfaces of a spinal or epidural catheter, or surgical drain entering or exiting the body especially those drains with a man-made entry point (percutaneous) that are draining a potentially contaminated space (ex. surgery drain post ruptured appendix). Efforts to render such percutaneous or indwelling medical devices microbially resistant have involved various dressings and protective antimicrobial coverings such as silver tipped catheters to protect from contamination during catheter placement, which has reduced but not eliminated infection. Antibiotics cannot sterilize an infected drain since it has no blood supply to deliver the medication. Use of antimicrobial light to disinfect such devices while they are in use provides a novel method of reducing the risk of nosocomial infections, but requires judicious stewardship of both human and environmental microbiomes, recalling that the latter refers to the combination of useful bacteria, virus and fungi collectively called microbiota, and their genomic contributions that mediate immunity, as well as antibiotic resistance where a well balanced microbiota is important for general health and wellness.

U.S. Pat. No. 10,245,424 discloses providing broadband UV sterilization of a fluid as it flows through a catheter or catheter connector. However, according to the '424 patent, a light reflector is disposed between the outer wall and inner wall of the connector body to reflect UV light into the interior of the connecting body, and no attempt is made to disinfect the external surface of a catheter or adjacent skin or tissues which is the primary route of pathogen access. That is not unexpected, since employing broadband UVC light (100-280 nm) as disclosed in the '424 patent could only be used on inanimate, unexposed surfaces to avoid exposing the patient or any healthcare worker to the toxicities of broad band UVC irradiance on eyes, skin or any adjacent tissue as this would result in potential damage to a patient or healthcare provider.

In accordance with the present disclosure safe antimicrobial wavelengths such as far UV-C wavelengths (200-230 nm) or safe visible antimicrobial light wavelengths are used to simultaneously disinfect indwelling medical devices such as catheters, vascular access lines, surgical drains, endotracheal or tracheostomy tubes, chest tubes, dialysis access catheters, feeding tubes and trocars while simultaneously disinfecting the patient's adjacent skin and tissues which can act as contamination or infection sources, or alternatively to disinfect such devices from inadvertent healthcare worker contamination during manipulation to curtail nosocomial infection. More particularly, in accordance with the present disclosure, we provide delivery of safe antimicrobial light to various percutaneous or indwelling medical devices while in use using fiber optic cables, optical fibers, light guides or light pipes for efficient transmission and delivery of such antimicrobial wavelengths from a photon source into small areas of such devices to simultaneously target animate and inanimate surfaces for disinfection in a near field location for optimal dosing efficacy.

Optical fibers, light guides and light pipes employ a property known as total internal reflection (TIR) to efficiently transmit light from a photon source to a target site with minimal loss of radiance. For example, in the case of fiber optic cables, light travels through the core of the cable which may be made up of multiple fibers or liquid constantly bouncing photons from the cladding (i.e., the mirror lined walls of the cable), by a principle called total internal reflection, and primarily exits the fiber optic cable or light guide only at the end of the cable. However, planned leakage of light can occur along the length of the cable or fiber in a fashion known as diffusion. Indeed, there are also commercially available side-emitting optical cables or optical fibers which emit light both through the side and the end of the cable. In accordance with the present invention, we employ both characteristics of light transmission/leakage of optical fibers, light guides, or light pipes cables to deliver antimicrobial wavelengths near field to a target to achieve disinfection of access sites prone to contamination and infection of percutaneous or indwelling medical devices such as catheters, vascular access lines, and surgical drains.

In accordance with one embodiment of this disclosure we provide percutaneous and indwelling medical devices such as urinary catheters, vascular catheters, spinal or epidural catheters or various surgical drains whether in body cavities or subcutaneous spaces, or endotracheal tubes, tracheostomy or chest tubes or trocars with longitudinal or helically shaped circumferential optical fibers or light pipes, or light guides with optical features to direct, refract, focus, select or diffuse safe antimicrobial light toward a defined target surface to deactivate target pathogens. The optical fibers, light pipes or light guides in turn are attached to a light source of safe antimicrobial light such as far UV-C light, or a safe antimicrobial visible light source. Alternatively, the percutaneous or indwelling medical device itself can also function as an optical fiber, i.e., as a light pipe or light guide. The light source may be either stationary such as a wall mounted unit in an ICU or hospital room or ambulance, or configured to be a mobile photon source attachable to a percutaneous or indwelling medical device to disinfect both internal and external surfaces of the indwelling medical devices as well as to deliver disinfecting irradiance to adjacent areas of the skin or tissues of the patient, and in particular at the catheter entry interface into the patient but also into tissues adjacent to the area for the full length of the device. The safe antimicrobial light can be continuous, intermittent, pulsed, automated, or manually activated to optimize deactivation of pathogens, while minimizing the dose of light to non-targeted skin or tissues, thereby minimizing irradiance of ambient environmental non target surfaces or health care workers in order to minimize the impact of such wavelengths on a patient's or health care workers individual or environmental microbiomes.

In one embodiment, the indwelling medical device comprises a urinary catheter formed of Teflon® coated latex, or silicon or hydrogel coated latex, or PVC (polyvinyl chloride) wrapped with, embedded with or co molded with optical fibers, or light pipes or light guides to transmit safe antimicrobial wavelengths in a near field location to both external and internal walls of the catheter for specific targets or intervals. These examples illustrate the potential embodiments but do not imply any limits or restrictions as to the material used for such medical devices.

In another aspect, the indwelling medical device comprises a vascular access catheter such as central or peripheral IV catheter, a PICC line, a dialysis access line, an arterial line, or a non-vascular access catheter such as Tenckoff peritoneal dialysis catheter or a spinal or epidural anesthesia catheter. The medical device in these examples contains a linearly or helically disposed optical fiber, light pipe or light guide configured to carry safe antimicrobial wavelengths to disinfect the skin/tissue-medical device interface and optionally may be used to disinfect a portion or the entire length of a device as well as adjacent tissues when desired. Optionally, the optical fiber cable, light pipe, or light guide may be configured to release antimicrobial wavelengths at intervals for specific targets, in near field locations, at specified intervals to minimize dosing requirements to achieve and maintain disinfection at the skin catheter interface or other target areas both animate and inanimate along the medical device while limiting antimicrobial irradiance to non-targeted animate or inanimate surfaces, thus protecting the natural microbiome. Alternatively, the indwelling medical device itself can also function as a light guide or light pipe with similar features.

In yet another embodiment, the indwelling medical device comprises a surgical drain such as a Jackson Pratt drain, a Hemovac drain or a negative pressure dressing or any other type of surgical drain, which is provided with a linear or circumferential helically configured optical fiber, light pipes or light guides to deliver antimicrobial wavelengths to disinfect the drain, on both interior and exterior surfaces thereof where the drain enters the patient's body and optionally along the entirety of or at intervals along the length of the drain, in particular targeting the tubing/skin interface where pathogens might migrate into the body to create infection—thereby eliminating the risk of ascending infection, but also allowing antimicrobial treatment of tissues adjacent to the drainage device when needed. The optical fibers, light pipes or light guides are configured to carry and deliver antimicrobial wavelengths which are safe for skin, tissue or ocular exposure, from a photon source to identified targets such as the tubing/skin interface and adjacent skin areas as well as along the length of the device inside the body. Alternatively, the indwelling medical device itself can also function as a light guide or light pipe.

In yet another embodiment, the percutaneous or indwelling medical device comprises an endotracheal or tracheostomy tube embedded with optical fibers, light pipes or light guides, either linearly configured or helically configured to radiate the circumference of such tubes both internally and externally with safe wavelengths of light to deactivate pathogens and prevent the transmission of pathogens along the length of such devices or at various intervals to targeted locations on the surfaces of such endotracheal tubes and surrounding skin or oral or nasal interface surfaces of the patient. Alternatively, the indwelling medical device itself can also function as a light guide or light pipe.

In still yet another embodiment, we provide fittings for the optical fibers, light tubes or light guides which are embedded or attached to urinary catheters, vascular catheter access lines, feeding tubes and surgical drains with connectors that are configured to transmit and deliver safe antimicrobial wavelengths from a central wavelength emitting photon source. The central wavelength photon source, such as a far UV-C excimer lamp, works in similar fashion to an electrical harness, wherein the central photon source of antimicrobial wavelengths serves as a manifold to connect to a plurality or network of multiple optical connectors which then connect to a plurality or network of optical fibers, light pipes or light guide such fibers, pipes or guides acting as conduits for distribution of disinfecting wavelengths in a near field location for efficient and targeted disinfection of various tubes, catheters, or drains for a single patient ensuring each potential source of nosocomial infection is routinely and actively disinfected while in use. The light source may be an excimer lamp, or an LED source of safe antimicrobial wavelengths such as far UV-C wavelengths (200-230 nm), that is stationary within a hospital room, or mobile and configured for use in an ambulatory capacity as in an ambulance, or on a patient who is mobile.

In still yet another embodiment, percutaneous or indwelling medical devices such as catheters, drainage tubes, endotracheal or tracheostomy tubes and those similarly functioning apparatus are fitted with optical fibers, light pipes, or light guides to transmit safe antimicrobial wavelengths from a light source, and deliver it in a near field location using optical filters within such optical fibers, pipes or guides features configured to focus the light on specific targets at specific dosing taking care to limit dosing to only what is necessary to achieve safe disinfection of specific pathogen targets such as a patient's skin/tubing or skin/catheter interface as well as specific targets within the device itself in order to limit ambient or environmental antimicrobial irradiance to avoid microbial disruption of human or environmental microbiomes. Alternatively, the indwelling medical device itself can also function as a light guide or light pipe with diffusion capabilities for specific targets.

In still yet another embodiment, indwelling medical devices such as catheters, drainage tubes, or endotracheal or tracheostomy tubes feeding tubes and the like are fitted with optical fibers, light pipes, or light guides configured to transmit safe antimicrobial wavelengths such as far UV-C or visible light to focus near field irradiance on specific pathogen targets such as skin/tubing or skin/tissue/catheter interface in order to limit the time and dose to achieve disinfection as well as to focus radiance on specific decontamination targets for individual human exposure, or to limit the wavelength exposure for healthcare workers working in the vicinity of treated patients, or limit exposure of other people or environments as a means to limit microbial disruption of a patient's or nearby individual's human microbiome or environmental microbiomes while at the same time preventing nosocomial infections.

More particularly, in one aspect A there is provided an apparatus maintaining disinfection or sterilization of percutaneous or indwelling medical devices configured for insertion into a patient's body, comprising a tube or similarly functioning form of medical grade material having a proximal end at the percutaneous entry and a distal end within the patient's body, wherein the drainage or delivery tube incorporates one or more fiber optic cables, optical fibers, light pipes or light guides extending from adjacent a proximal end of the tube towards a distal end of the tube configured to direct and deliver safe antimicrobial wavelengths near field (<10 cm and preferably <1 cm) from source to both animate and inanimate targets, such wavelengths preferably include far UV-C (200-230 nm) to safely and efficiently disinfect the medical device and adjacent patient tissue at targeted locations while in use with a optimally efficient dosing to reduce nosocomial infections while protecting the human and environmental microbiome.

In such aspect A the optical fibers, light pipe or light guide may be configured with optical features selected from variable cladding and reflective materials to mediate refraction, filters or micro prisms to mediate direction, or spiral or various shaped grooves lasered into the fibers to create diffusion, buffering modalities include fiber delay lines, each added onto, or manufactured as part of the optical fibers, light tubes or light guides which are then molded onto or into such devices to refract, diffuse, reflect, focus or direct antimicrobial light on either or both external and internal surfaces of a device or along its length as well as into adjacent targeted patient tissues to aid in preventing or reducing pathogen burden in the case of an already contaminated device.

In aspect A the optical features may be configured to directionally reverse antimicrobial light direction inside an optical fiber, light tube, or light guide such that light within an IV or other catheters and drains can radiate the skin puncture both from outside and inside a percutaneous wound site in order to disinfect the puncture site and prevent ascending infection further along the device.

In aspect A the optical fiber, light pipe or light guide may be configured to confine, direct, diffuse, reflect, refract, or diffusely emit or focus antimicrobial wavelengths of light at selected positions along its length.

In aspect A the fiber optic cable, optical fiber, light pipe or light guide may have surface features configured with optical features that intentionally diffuse, refract, reflect, or focus antimicrobial wavelengths of light inwardly and/or outwardly and/or at a focused target backwardly from the sides or distal end of the optical fibers, light pipe or light guide to direct antimicrobial light to specific targets to reduce the risk of infection or reduce pathogen burden when contaminants are already present.

In aspect A the apparatus may further include an antimicrobial light source safe for patient and health care worker exposure, wherein the light is far UV-C and has a wavelength of 200-230 nm, or a blue visible light source having a wavelength of 400-470 nm or any antimicrobial visible wavelengths as may be identified safe and effective for human or animal exposure and provides ongoing disinfection of the device and adjacent tissues while the device is in use.

In aspect A the percutaneous or indwelling medical device may be selected from the group consisting of a urinary system catheters (nephrostomy, suprapubic, urethral, bladder), a vascular access line (intravenous or arterial), spinal or epidural anesthesia catheters, a peritoneal or vascular dialysis catheter an endotracheal or tracheostomy tube, chest tubes, feeding tubes, a trocar or any percutaneous surgical drains which enter any body cavity spaces, orthopedic spaces or subcutaneous spaces.

In aspect A the optical features may be selected from variable cladding and reflective materials to mediate refraction, filters or micro prisms to mediate direction, or spiral or various shaped grooves lasered into the fibers to create diffusion, buffering modalities include fiber delay lines, each added onto, or manufactured as part of the optical fibers, light tubes or light guides which are then molded onto or into such devices to refract, diffuse, reflect, focus or direct antimicrobial light on either or both external and internal surfaces of a device or along its length as well as into adjacent targeted patient tissues to aid in preventing or reducing pathogen burden in the case of an already contaminated device.

In aspect A the catheter may comprise a urethral catheter, a PICC line or a vascular dialysis line.

In aspect A, the apparatus may comprise multiple fiber optic cables, optical fibers, light tubes, or light guides for delivery of antimicrobial wavelengths in a near field location (preferably <10 cm, more preferably <1 cm) to a specified target for optimally effective dosing allowing for shorter periods of irradiance to provide effective disinfection of target pathogens while protecting the human and environmental microbiome from dysbiosis.

In aspect A, the apparatus may comprise single or multiple fiber optic cables, optical fibers, light tubes, or light guides which may be an ‘add on’ or wrap around to an existing medical device or the medical device and optical fibers, light tubes or light guides are manufactured to be co-molded or embedded into as part of the device.

In aspect A, the fiber optic cable, optical fibers, light tube or light guide may be helically wound onto the medical device or manufactured to be co molded in a helical configuration within the medical device.

In aspect A the fiber optic cable or single or multiple optical fiber, light tube or light guide may comprise a side-emitting device or may provide openings configured for diffusion and dispersion of a particular dose of light along its length as well as its distal end.

In aspect A the catheter tube or drain itself may further include a polarizing layer along its entirety or a part thereof, wherein the polarizing layer preferably comprises a linear, elliptical or a circular polarizing configuration.

In aspect A the light source may include a central safe antimicrobial photon source attached to an optical harness which acts as a manifold emitting safe antimicrobial light wavelengths, preferably UVC wavelengths, (200-230 nm) configured to deliver the light via a plurality of optical connectors attached by a single connector to each of one or more fiber optic cables, optical fibers, light tubes or light guides attached to or within percutaneous or indwelling medical devices to simultaneously or consecutively provide antimicrobial disinfecting wavelengths for multiple different percutaneous or indwelling medical devices used in care of a patient such as urinary catheters, spinal or epidural catheters, various types of vascular access catheters, dialysis vascular or peritoneal access catheters, surgical drains into body cavity spaces, orthopedic spaces or joints or subcutaneous spaces, trocars, endotracheal or tracheostomy tubes, chest tubes, intracranial pressure monitors, each device having been manufactured or co molded or attached with one or more fiber optic cables, optical fibers, light tubes or light guides to connect to the central photon source and deliver such antimicrobial wavelengths in a near field location to intended targets such as the percutaneous entry point, or within the medical device itself and to adjacent potentially contaminated patient tissues or spaces.

In aspect A, the apparatus may include an optical fiber, light tube or light guide emanating from the photon source where the source is fitted with an optical twist connector which then connects to a length of fiber optic cable or optical fibers from 1-5 m in length that offers a plurality of connectors at its distal end to connect to other individual optical fiber connectors that then deliver antimicrobial wavelengths to different devices again using a twist connector from the source to an extension fiber optic cable, optical fiber, light tube or light guide which then attaches via a connector such as a twist or other types of optical connectors to the optical fiber, light tube or light guide which is embedded in or attached to the various percutaneous or indwelling catheters, drains, endotracheal or other tubes, or trocar assembly, so that two connections and connectors are required to deliver the desired wavelengths at a specified dose to the medical devices—the harness extension being a re-useable connector. In the absence of the optical harness extension each device would require its own 1-5 m fiber optic, light tube or light guide extension to connect to the central photon source which would need to be disposed of after use.

In aspect A the central antimicrobial light source may be configured for an automatic, or manual or electronic or mechanical controller, or is programmable for continuous, intermittent, or pulsed or for variable intensity dosing in a near field location to specific targets to achieve optimal antimicrobial efficacy with a lowest targeted dose of effective irradiance in order to minimize the impact on human and environmental microbiomes.

In aspect A the light source may include a linear or circular light polarizer to achieve optimal desired wavelengths and control the flow of light.

According to aspect B there is provided an indwelling or percutaneous medical device configured for insertion into a patient's body, comprising a tube or similarly functioning configuration for fluid delivery, physiologic monitoring, or physiologic drainage is formed of medical grade material having a proximal end and a distal end, wherein an internal surface of the tube is covered at least in part with a light reflecting material, whereupon the device is configured to carry both a fluid and antimicrobial light delivery source such as optical fiber, light pipe or light guide along its length, or at least in part.

In aspect B, the light reflective material may be at least partially reflective to far UV-C light having a wavelength of 200-230 nm, or blue light having a wavelength of 400-470 nm.

In aspect B the tube or device may act an optical fiber, light tube or light guide to include optical features selected from variable cladding and reflective materials to mediate refraction, filters or micro prisms to mediate direction, or spiral or various shaped grooves lasered into the fibers to create diffusion, buffering modalities include fiber delay lines, each added onto, or manufactured as part of the optical fibers, light tubes or light guides which are then molded onto or into such devices to refract, diffuse, reflect, focus or direct antimicrobial light on either or both external and internal surfaces of a device or along its length as well as into adjacent targeted patient tissues to aid in preventing or reducing pathogen burden in the case of an already contaminated device on its outer and or inner surface.

In aspect B the medical device may be selected from the group consisting of various percutaneous urinary drainage, spinal or epidural monitoring or delivery catheters, an indwelling vascular access or monitoring line, an endotracheal, tracheostomy, chest tube, feeding tubes a trocar and any surgical drain that enters a body cavity or space, orthopedic spaces, or subcutaneous spaces.

In aspect B the device may further comprise a central disinfecting light source configured to generate safe antimicrobial light which acts as a manifold to deliver antimicrobial wavelengths near field to such devices as well as to adjacent animate and inanimate targets using optical fibers, light pipes or light guides to prevent nosocomial infections or reduce contaminant burdens in various physiologic spaces.

In aspect B the light source may be configured to generate antimicrobial far UV-C light having a wavelength of 200-230 nm, or blue light having a wavelength of 400-470 nm.

### BRIEF DESCRIPTION OF THE DRAWINGS

Further features and advantages of the subject disclosure will be seen from the following detailed description, taken in conjunction with the accompanying drawings, wherein like numerals depict like part, and wherein:

FIG. 1 schematically illustrates an IV catheter and tubing embedded with optical fibers configured for delivering antimicrobial light in accordance with a first embodiment of the disclosure;

FIG. 2 schematically shows the catheter of FIG. 1 affixed to the skin of a patient;

FIG. 2A is a side view of the catheter of FIG. 1;

FIG. 3 schematically illustrates a drain with embedded optical fibers transmitting far UV-C light (200-230 nm) or any visible antimicrobial wavelengths in accordance with another embodiment of the present disclosure;

FIG. 4 schematically illustrates a urinary catheter having optical fibers or light guides configured to emit antimicrobial wavelengths in accordance with the present disclosure;

FIG. 5 schematically illustrates a urethral catheter of FIG. 4, in a bladder; and

FIGS. 6-8A illustrate various arrangements of tubular medical devices carrying optical fibers, light pipes, or light guides for distribution of safe antimicrobial wavelengths in accordance with present disclosure.

### DETAILED DESCRIPTION

As used herein the terms “fiber optic cable or cables”, “light pipe”, “light guide”, and “optical fibers” are used interchangeably.

As used herein “indwelling medical device” is intended to include any type of medical device with hollow or tubular characteristics configured for insertion into the body, including but not limited to catheters, drains, endotracheal or tracheostomy tubes, vascular access devices and trocars. By way of example but not limitation, catheters are manufactured for various applications such as cardiovascular, urological, gastrointestinal, neurologic, and ophthalmic procedures. Catheters can comprise thin hollow tubes typically configured for insertion into a body cavity, orifice, duct or vessel, skin, or adipose tissue. Catheters are designed to be left inside the body either temporarily, or permanently and functionally may allow for drainage, administration of fluids or gases, access by surgical instruments, or to provide a wide variety of medical tasks depending on the type of catheter or tube. Special types of catheters, sometimes called “probes”, may be used in preclinical or clinical sampling of fluids or tissues.

As used herein “safe antimicrobial light” is intended to include light that exhibits antimicrobial activity but is considered to be safe for skin and tissue exposure, and optionally safe for ocular exposure.

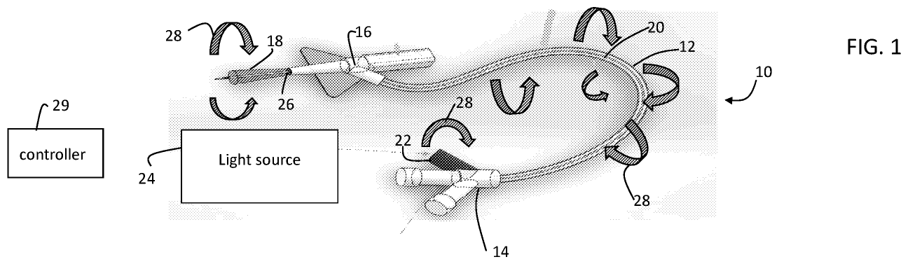

FIG. 1 illustrates a catheter 10 typically used for intravenous therapy, e.g., for administration of fluids or medications and nutrients directly into a patient's vein. Catheter 10 includes an elongated hollow tube 12 formed of a medical grade flexible polymeric material. Catheter 10 includes a housing 16 configured to hold a needle 18 at its distal end. In this illustration a coaxial fiber optic cable 20 is provided running the length of tube 12 from the proximal to distal end of the catheter. Fiber optic cable 20 may comprise a light guide or light pipe inside clear tubing that radiates the light filtered to emit filtered UVC light (200-230 nm), or other antimicrobial light back onto tubing or onto skin or surrounding tissue catheter interface, which limits ambient radiance to limit effect on microbiome. A fiber optic connector 22 is affixed to one end of the fiber optic cable 20 at the proximal end 14 and configured to be attached to a safe antimicrobial light source 24. Light source 24 may comprise a far UV-C (200-230 nm) light source, or a visible antimicrobial light source that is safe for human skin and tissue exposure such as blue light of 400-470 nm wavelength. A concave lens 26 is provided at the distal end of fiber optic cable 20 and configured to direct wavelength irradiation onto needle 18 and also to diffuse light in a targeted fashion out of housing 16. Housing 16 is made of a clear polymeric material such as polycarbonate with integrated lens which produces a light source coaxial with needle and high intensity illumination at puncture site and omnidirectional diffuse emission out of entire housing in all directions, and is transmissive to wavelengths in at least the 200-230 nm (far UV-C) and/or the 400-470 nm wavelengths. The optical fibers, light pipes, or light guides direct light waves back into the catheter tubing and skin/catheter interface as indicated by arrows 28. Fiber optical cable 20 may also be designed to leak, diffuse, or emit light wavelengths along selected and targeted locations as will be described below. Controller 29 controls the light source emission on intervals and/or varies intensity over time.

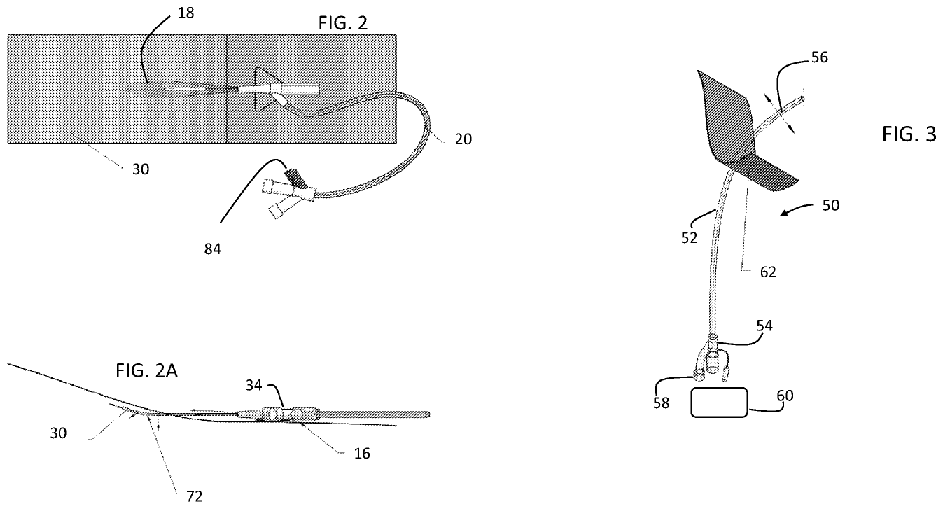

FIGS. 2 and 2A illustrate the catheter of FIG. 1 with the needle 18 inserted into the skin 30 and a cone of UVC light illuminates the needle and IV entry into the skin/vein of a patient. Referring in particular to FIG. 2A, as can be seen, the fiber optic cable 20 (shown in FIG. 1) is configured to emit antimicrobial wavelengths back onto the catheter tubing and the skin catheter interface, but due to its internal reflective coating avoids radiating the ambient environment to protect the microbiome. Also, as seen in FIG. 2A, the needle housing 16 (shown in FIG. 1) includes an interior reflective coating 34 so that the needle housing 16 also functions as an antimicrobial wavelengths light pipe and carries light from the fiber optic cable 20 through housing and coaxially omnidirectionally diffusing along the needle 18 (shown in FIG. 2) into the patient tissue in targeted directions, using filters and openings.

The light source 24 may comprise a light source of safe antimicrobial light, more particularly, a far UV-C light source (200-230 nm), or visible antimicrobial blue light source (400-470 nm), such as an excimer light or LED light. Far UV-C light primarily exhibits antimicrobial activity by directly damaging the genetic material of a pathogen but is considered safe for skin and tissue exposure as well as ocular exposure because it is not absorbed by these structures. Blue light primarily exhibits antimicrobial activity through activation of androgenous photo synthesizers, which leads to formation of reactive oxygen species that attack components of bacterial cells. Blue light is considered innocuous to the skin, but potentially may inflict photo damage to the eyes. Accordingly, if the light source is a blue light source, measures should be taken to prevent the light from shining outwardly or from ocular exposure, i.e., away from the skin of the patient. This can be accomplished by exterior shielding of the proximal end of the catheter, i.e., so that antimicrobial light may radiate surfaces internal to the patient but is prevented from shining outwardly from the skin of the patient.

FIG. 3 illustrates a surgical drain configured with an optical fiber system for delivering antimicrobial light in accordance with the present disclosure. Drain 50 comprises an elongated hollow tube 52 formed of a medical grade flexible polymeric material. Drain 50 includes a connector 54 at its proximal end. An optical fiber 56 is provided within tube 52 running the length of tube 52. Optical fiber 56 is connected at its proximal end 58 to an antimicrobial light source 60. As before, antimicrobial light source 60 may comprise a far UV-C wavelength (200-230 nm) source or a visible antimicrobial light source such as a blue light source. If the light source comprises blue light, which is innocuous to skin but may affect photo damage to the eyes, optical fiber 56 internal coating should be continuous at visibly exposed surfaces to minimize blue light from escaping along its length of the cable up to the point where the drain is introduced into the patient tissue/skin 62.

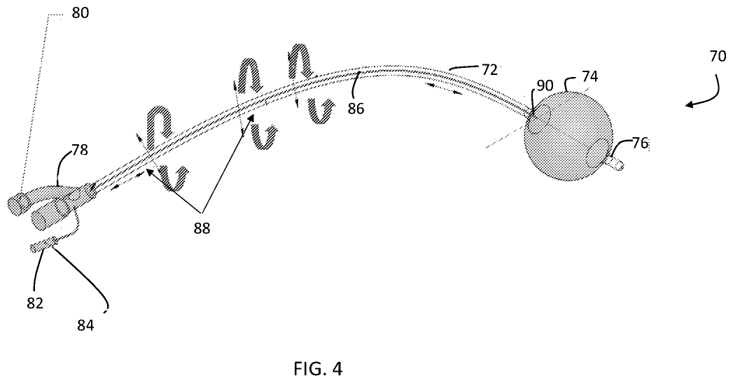

FIG. 4 illustrates a urethral catheter 70 in accordance with the present disclosure. Urethral catheter 70 comprises an elongate optically clear flexible tube 72 formed of a medical grade material having a balloon 74 adjacent its distal end 76. A connector 78 at the proximal end of catheter 70 includes a luer fitting 80 and an optical fiber connector 82 which in turn is connected to a remote antimicrobial light source 84 which travels down the catheter circumferentially and leaves fiber at specified locations at the entrance to urethra, and emits far UVC (200-230 nm), or other antimicrobial light reflects back on itself to catheter, or focused on surrounding tissue that touches the catheter, such as a urethral opening.

A flexible side-emitting optical fiber 86 is provided inside or embedded into tube 72 running from optical fiber connector 82 to balloon 74. As before, antimicrobial light source 84 may comprise a far UV-C light source, or a blue light source or any visible safe and effective antimicrobial wavelength source. If the light source comprises a blue light source, optical fiber 86 internal coating should be continuous along its length designed to be outside the body to minimize blue light from escaping up to the point where the catheter is introduced into the urethra. In use, antimicrobial light travels down catheter circumferentially and may leave optical fiber at specific locations 88, and at the entrance to the urethra 90.

Various changes may be made from the foregoing disclosure without departing from the spirit and the scope thereof. For example, as illustrated in FIG. 5, a cannula 100 may be coated on its inside walls 102 with reflective material so that the catheter itself also function as an optical fiber, light pipe or light guide when connected to a far UV-C light source or other antimicrobial light source (not shown) to radiate safe antimicrobial light in accordance with the present disclosure.

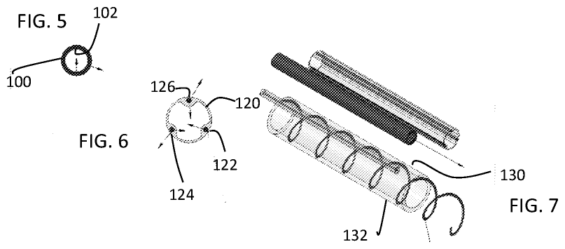

Also, and with reference to FIG. 6, a catheter tube 120 may be provided with multiple co-molded optical fibers and serves as an optical guide, light pipes or light guides 122, 124, 126.

In still yet another embodiment of the disclosure, illustrated in FIG. 7, the optical fiber 130 may comprise a spiral shape running the length of a hollow medical device such as a catheter or a trocar 132.

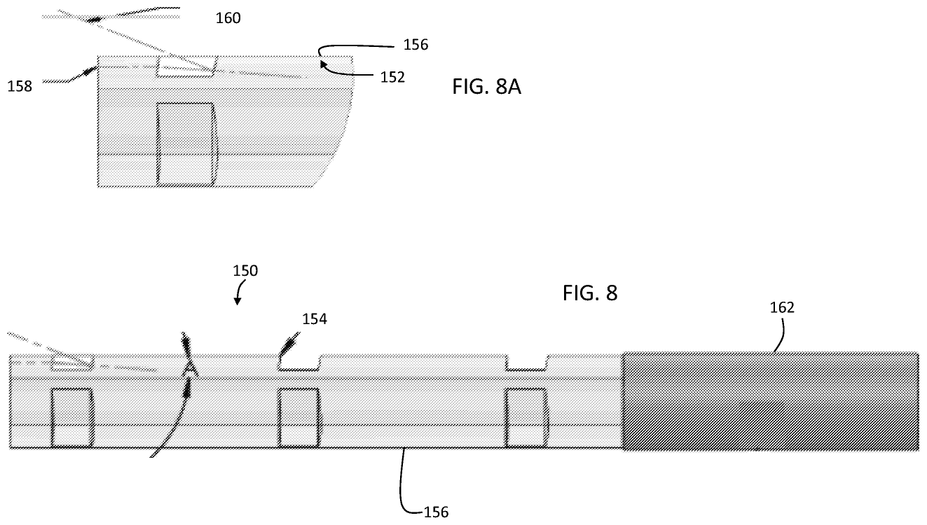

In yet another embodiment illustrated in FIGS. 8 and 8A, a hollow medical device such as a catheter 150 is provided with a light reflective coating 152 on its inner wall so as to also function as an optical fiber, and has a plurality of optical features such as facets 154 formed, e.g., by molding or machining on its outer wall 156 whereby to permit light rays 158 to reflect, refract, diffuse or focus light off the walls of the facets 154, and reflect light back out as light rays 160. Catheter 150 may also be provided with a light blocking coating such as a polarizing layer 162 along selected portions of its length to prevent light emission for microbiome protection and/or ocular protection in the case of a blue light source. Polarizing layers 162 may be chemical or plated and comprise a linear or circular polarization function that blocks most of the light emission into the room but allows the light to shine on the patient's skin.

In yet another embodiment, the proximal end of the optical fiber may be coterminous with the proximal end of the catheter and permanently fixed to the proximal end of the catheter, for direct connection to a light source on the wall or on the patient's bed or for an ambulatory light source if the patient is ambulatory. Still other changes are possible.

What is claimed:

1. An apparatus for maintaining disinfection or sterilization of percutaneous or indwelling medical devices configured for insertion into a patient's body, comprising a tube or similarly functioning form of medical grade material having a proximal end at the percutaneous entry and a distal end within the patients body, wherein the drainage or delivery tube incorporates one or more fiber optic cables, optical fibers, light pipes or light guides extending from adjacent a proximal end of the tube towards a distal end of the tube configured to direct and deliver safe antimicrobial wavelengths near field (<10 cm and preferably <1 cm) from source to both animate and inanimate targets, such wavelengths preferably include far UV-C (200-230 nm) to safely and efficiently disinfect the medical device and adjacent patient tissue at targeted locations while in use with a optimally efficient dosing to reduce nosocomial infections while protecting the human and environmental microbiome.

2. The apparatus of claim 1, wherein the optical fibers, light pipe or light guide are configured with optical features selected from variable cladding and reflective materials to mediate refraction, filters or micro prisms to mediate direction, or spiral or various shaped grooves lasered into the fibers to create diffusion, buffering modalities include fiber delay lines, each added onto, or manufactured as part of the optical fibers, light tubes or light guides which are then molded onto or into such devices to refract, diffuse, reflect, focus or direct antimicrobial light on either or both external and internal surfaces of a device or along its length as well as into adjacent targeted patient tissues to aid in preventing or reducing pathogen burden in the case of an already contaminated device.

3. The apparatus of claim 2, wherein the optical fiber, light pipe or light guide is configured to confine, direct, diffuse, reflect, refract, or diffusely emit or focus antimicrobial wavelengths of light at selected positions along its length.

4. The apparatus of claim 1, wherein the fiber optic cable, optical fiber, light pipe or light guide has surface features configured with optical features that intentionally diffuse, refract, reflect, or focus antimicrobial wavelengths of light inwardly and/or outwardly and/or at a focused target backwardly from the sides or distal end of the optical fibers, light pipe or light guide to direct antimicrobial light to specific targets to reduce the risk of infection or reduce pathogen burden when contaminants are already present.

5. The apparatus of claim 1, further including an antimicrobial light source safe for patient and health care worker exposure, wherein the light is far UV-C and has a wavelength of 200-230 nm, or a blue visible light source having a wavelength of 400-470 nm or any antimicrobial visible wavelengths as may be identified safe and effective for human or animal exposure and provides ongoing disinfection of the device and adjacent tissues while the device is in use.

6. The apparatus of claim 1, wherein the percutaneous or indwelling medical device is selected from the group consisting of a urinary system catheters (nephrostomy, suprapubic, urethral, bladder), an vascular access line (intravenous or arterial), spinal or epidural anesthesia catheters, a peritoneal or vascular dialysis catheter an endotracheal or tracheostomy tube, chest tubes, feeding tubes, a trocar or any percutaneous surgical drains which enter any body cavity spaces, orthopedic spaces or subcutaneous spaces.

7. The apparatus of claim 6, wherein the optical features are configured to directionally reverse antimicrobial light direction inside an optical fiber, light tube or light guide such that light within an IV or other catheters and drains can radiate the skin puncture both from outside and inside a percutaneous wound site in order to disinfect the puncture site and prevent ascending infection further along the device.

8. The apparatus of claim 6, wherein the catheter comprises a urethral catheter, a PICC line or a vascular dialysis line.

9. The apparatus of claim 1, comprising multiple fiber optic cables, optical fibers, light tubes, or light guides for delivery of antimicrobial wavelengths in a near field location (preferably <10 cm, more preferably <1 cm) to a specified target for optimally effective dosing allowing for shorter periods of irradiance to provide effective disinfection of target pathogens while protecting the human and environmental microbiome from dysbiosis.

10. The apparatus of claim 1, wherein single or multiple fiber optic cables, optical fibers, light tubes or light guides may be an ‘add on’ or wrap around to an existing medical device or the medical device and optical fibers, light tubes or light guides are manufactured to be co-molded or embedded into as part of the device.

11. The apparatus of claim 1, wherein the fiber optic cable, optical fibers, light tube, or light guide is helically wound onto the medical device or manufactured to be co-molded in a helical configuration within the medical device.

12. The apparatus of claim 1, wherein the fiber optic cable or single or multiple optical fiber, light tube or light guide comprises a side-emitting device or may provide openings configured for diffusion and dispersion of a particular dose of light along its length as well as its distal end.

13. The apparatus of claim 10, where the catheter tube or drain itself, further includes a polarizing layer along its entirety or a part thereof, wherein the polarizing layer preferably comprises a linear, elliptical or a circular polarizing configuration.

14. The apparatus of claim 5, wherein the light source includes a central safe antimicrobial photon source attached to an optical harness which acts as a manifold emitting safe antimicrobial light wavelengths, preferably UV-C wavelengths, (200-230 nm) configured to deliver the light via a plurality of optical connectors attached by a single connector to each of one or more fiber optic cables, optical fibers, light tubes or light guides attached to or within percutaneous or indwelling medical devices to simultaneously or consecutively provide antimicrobial disinfecting wavelengths for multiple different percutaneous or indwelling medical devices used in care of a patient such as urinary catheters, spinal or epidural catheters, various types of vascular access catheters, dialysis vascular or peritoneal access catheters, surgical drains into body cavity spaces, orthopedic spaces or joints or subcutaneous spaces, trocars, endotracheal or tracheostomy tubes, chest tubes, intracranial pressure monitors, each device having been manufactured or co-molded or attached with one or more fiber optic cables, optical fibers, light tubes or light guides to connect to the central photon source and deliver such antimicrobial wavelengths in a near field location to intended targets such as the percutaneous entry point, or within the medical device itself and to adjacent potentially contaminated patient tissues or spaces.

15. The apparatus of claim 5, where an optical fiber, light tube or light guide emanating from the photon source where the source is fitted with an optical twist connector which then connects to a length of fiber optic cable or optical fibers from 1-5 m in length that offers a plurality of connectors at its distal end to connect to other individual optical fiber connectors that then deliver antimicrobial wavelengths to different devices again using a twist connector from the source to an extension fiber optic cable, optical fiber, light tube or light guide which then attaches via a connector such as a twist or other types of optical connectors to the optical fiber, light tube or light guide which is embedded in or attached to the various percutaneous or indwelling catheters, drains, endotracheal or other tubes, or trocar assembly, so that two connections and connectors are required to deliver the desired wavelengths at a specified dose to the medical devices—the harness extension being a re-useable connector thereby eliminating the requirement that each device have its own 1-5 m fiber optic, light tube or light guide extension to connect to the central photon source which would need to be disposed of after use.

16. The apparatus of claim 5, wherein the central antimicrobial light source is configured for an automatic, or manual or electronic or mechanical controller, or is programmable for continuous, intermittent, or pulsed or for variable intensity dosing in a near field location to specific targets to achieve optimal antimicrobial efficacy with a lowest targeted dose of effective irradiance in order to minimize the impact on human and environmental microbiomes.

17. The apparatus of claim 5, wherein the light source includes a linear or circular light polarizer to achieve optimal desired wavelengths and control the flow of light.

18. An indwelling or percutaneous medical device configured for insertion into a patient's body, comprising a tube or similarly functioning configuration for fluid delivery, physiologic monitoring, or physiologic drainage is formed of medical grade material having a proximal end and a distal end, wherein an internal surface of the tube is covered at least in part with a light reflecting material, whereupon the device is configured to carry both a fluid and antimicrobial light delivery source such as optical fiber, light pipe or light guide along its length, or at least in part.

19. The indwelling or percutaneous medical device of claim 18, wherein the light reflective material is at least partially reflective to far UV-C light having a wavelength of 200-nm, or blue light having a wavelength of 400-470 nm.

20. The indwelling or percutaneous medical device of claim 18, wherein the tube or device acts an optical fiber, light tube or light guide to include optical features selected from variable cladding and reflective materials to mediate refraction, filters or micro prisms to mediate direction, or spiral or various shaped grooves lasered into the fibers to create diffusion, buffering modalities include fiber delay lines, each added onto, or manufactured as part of the optical fibers, light tubes or light guides which are then molded onto or into such devices to refract, diffuse, reflect, focus or direct antimicrobial light on either or both external and internal surfaces of a device or along its length as well as into adjacent targeted patient tissues to aid in preventing or reducing pathogen burden in the case of an already contaminated device on its outer and or inner surface.

21. The indwelling or percutaneous medical device of claim 18, wherein the medical device is selected from the group consisting of various percutaneous urinary drainage, spinal or epidural monitoring or delivery catheters, an indwelling vascular access or monitoring line, an endotracheal, tracheostomy, chest tube, feeding tubes a trocar and any surgical drain that enters a body cavity or space, orthopedic spaces, or subcutaneous spaces.

22. The indwelling or percutaneous medical device of claim 18, further comprising a central disinfecting light source configured to generate safe antimicrobial light which acts as a manifold to deliver antimicrobial wavelengths near field to such devices as well as to adjacent animate and inanimate targets using optical fibers, light pipes or light guides to prevent nosocomial infections or reduce contaminant burdens in various physiologic spaces.

23. The indwelling or percutaneous medical device of claim 22, wherein the light source is configured to generate antimicrobial far UV-C light having a wavelength of 200-230 nm, or blue light having a wavelength of 400-470 nm.
